(19) United States
(12) Patent Application Publication
(10) Pub. No.: US 2026/0325834 A1
(43) Pub. Date: Oct. 22, 2026

(54) TEMPORARY PACING LEAD MANAGEMENT SYSTEM

(71) Applicant: Medtronic, Inc., Minneapolis, MN (US)

(72) Inventors: Mikayle A. Holm, Saint Anthony, MN (US); Kevin R. Seifert, Forest Lake, MN (US); Jonathan A. Hughes, Blaine, MN (US); Teresa A. Whitman, Dayton, MN (US); Thomas A. Wong, Little Canada, MN (US); Andrea J. Asleson, Maple Grove, MN (US)

(21) Appl. No.: 19/114,742

(22) PCT Filed: Sep. 18, 2023
(86) PCT No.: PCT/US2023/074481

§ 371 Date: Mar. 24, 2025

Related U.S. Application Data
(60) Provisional application No. 63/377,135, filed on Sep. 26, 2022.

(51) Int. Cl. A61N 1/05; A61N 1/362; A61N 1/375

(52) U.S. Cl. CPC A61N 1/057; A61N 1/362; A61N 1/37518

(57) ABSTRACT

A system including an electrical lead, an implantable medical device, wherein the electrical lead is configured to be electrically connected to the implantable medical device, and the implantable medical device is configured to deliver an electrical therapy to tissue of a patient via the electrical lead, a sheath configured to be at least partially disposed within vasculature of the patient and configured to receive the electrical lead such that a distal portion of the electrical lead is placed within the vasculature through the sheath, and a lead management device connected to the implantable medical device and the sheath, wherein the lead management device is configured to secure an excess portion of the electrical lead, and wherein the lead management device is configured to control movement of the electrical lead within the vasculature of the patient.

This application claims the benefit of U.S. Provisional Patent Application No. 63/377,135, filed Sep. 26, 2022 and entitled “TEMPORARY PACING LEAD MANAGEMENT SYSTEM,” the entire contents of which is incorporated herein by reference.

### TECHNICAL FIELD

This disclosure relates to medical device systems and, more particularly, to medical device systems for delivery of electrical stimulation therapy.

### BACKGROUND

In some situations, medical professionals may insert an implantable medical lead into the body of the patient to deliver various therapies. In some examples, the implantable medical leads may be temporary and may be removed from the patient after a period of time. In some examples, with respect to cardiac-related issues, a medical professional may insert a type of implantable medical lead also referred to as a temporary pacing lead into a heart of the patient and deliver temporary cardiac pacing to the heart.

### SUMMARY

The devices, systems, and techniques of this disclosure generally relate to managing movement of an implantable medical lead within a patient. In some examples, the implantable medical lead may have an excess portion that may be placed near an implantable medical device connected to the implantable medical lead. A clinician may need to control movement and/or length of the implantable medical lead within the patient. In some examples, the clinician may need to remove the implantable medical device and the implantable medical lead from the patient. This disclosure describes devices, systems, and techniques for managing movement of the implantable medical lead within the patient. This disclosure further describes devices, systems, and techniques for facilitating removal of the implantable medical lead from the patient.

In an example, the disclosure describes a system comprising: an electrical lead; an implantable medical device, wherein the electrical lead is configured to be electrically connected to the implantable medical device, and the implantable medical device is configured to deliver an electrical therapy to tissue of a patient via the electrical lead; a sheath configured to be at least partially disposed within vasculature of the patient and configured to receive the electrical lead such that a distal portion of the electrical lead is placed within the vasculature through the sheath; and a lead management device connected to the implantable medical device and the sheath, wherein the lead management device is configured to secure an excess portion of the electrical lead, and wherein the lead management device is configured to control movement of the electrical lead within the vasculature of the patient.

In another example, the disclosure describes a lead management device comprising: a first pocket configured to retain an implantable medical device; and a second pocket configured to retain an excess portion of an electrical lead, wherein the electrical lead is electrically connected to the implantable medical device, and wherein the electrical lead is configured to be disposed within a vasculature of a patient and to deliver a medical therapy to tissue of the patient.

In another example, the disclosure describes a lead management device comprising: an elongated body defining: a central groove extending from a proximal end of the elongated body to a distal end of the elongated body, wherein the central groove is configured to retain a first portion of an electrical lead connected to an implantable medical device; and a plurality of grooves disposed around the central groove and configured to retain a second portion of the electrical lead, wherein the lead management device is configured to control movement of the electrical lead.

In another example, the disclosure describes a method comprising: inserting a sheath and a distal portion of an electrical lead disposed within the sheath into vasculature of a patient; navigating the distal portion of the electrical lead to a target location within the vasculature of the patient; implanting the distal portion of the electrical lead in tissue at the target location; disposing an excess portion of the electrical lead within a lead management device, wherein the lead management device controls movement of the electrical lead; and delivering medical therapy to the tissue at the target location through the electrical lead.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device system including a lead management system.

FIG. 2 is a block diagram illustrating an example configuration of an implantable medical device of the system of FIG. 1.

FIG. 3A is a conceptual diagram illustrating an example sheath of an example lead management system.

FIG. 3B is a conceptual diagram illustrating a cross-section diagram of the sheath of FIG. 3A taken along line A-A.

FIG. 3C is a conceptual diagram illustrating the sheath of FIG. 3B with an electrical lead.

FIG. 4A is a conceptual diagram illustrating another example lead management device.

FIG. 4B is a conceptual diagram illustrating a cross-section view of the lead management device of FIG. 4A taken along line B-B.

FIG. 4C is a conceptual diagram illustrating an example medical device system with the lead management device of FIG. 4A.

FIG. 4D is a conceptual diagram illustrating another example medical device system with another example lead management device of FIG. 4A.

FIG. 5A is a conceptual diagram illustrating another example lead management system.

FIG. 5B is a conceptual diagram illustrating an example retainer of FIG. 5A.

FIG. 5C is a conceptual diagram illustrating another example retainer of FIG. 5A.

FIG. 6 is a flowchart illustrating an example process of implanting an medical device system in a patient.

### DETAILED DESCRIPTION

Medical devices, systems, and techniques of this disclosure relates to the managing movement of an implantable medical lead within a patient and facilitating removal of the implantable medical lead from the patient. A medical professional may insert an implantable medical lead into the patient (e.g., through vasculature of the patient) to deliver electrical signals (e.g., for purposes of medical therapy, stimulation, or the like) to tissue of the patient. The implantable medical lead (also referred to herein as “electrical lead”) may be connected to an implantable medical device. The implantable medical device may generate the electrical signals and transmit the electrical signals to the tissue of the patient through one or more implantable medical leads.

In some examples, based on the anatomy of the patient and/or the target location, the length of the implantable medical lead may be longer than necessary to reach the target site from the implantable medical device, and the clinician may store the excess lead lengths in subcutaneous tissue of the patient (e.g., adjacent to the implantable medical device within the subcutaneous tissue). Storage of the excess lead lengths in the tissue of the patient may lead to twisting, crimping, and/or pinching of the implantable medical lead which may negatively affect performance of the lead. In some examples, tissue may grow around the implantable medical device and/or the excess lead lengths within the subcutaneous tissue and renders removal of the implantable medical device and/or the implantable medical lead difficult and time consuming.

This disclosure provides improvements over other implantable medical lead systems and insertion processes by allowing a clinician to manage the length of an implantable medical lead within the vasculature of the patient and/or to manage movement of the implantable medical lead within a subcutaneous pocket in the tissue of the patient and/or within the vasculature of the patient. The medical devices, systems, and techniques of this disclosure also isolates the excess portions of the implantable medical lead stores in the subcutaneous pocket from tissue of the patient and facilitates removal of the implantable medical device and the implantable medical lead during a later operation. The medical devices, systems, and techniques of this disclosure may also enable identification of an orientation of the distal end of the implantable medical lead in addition to its position.

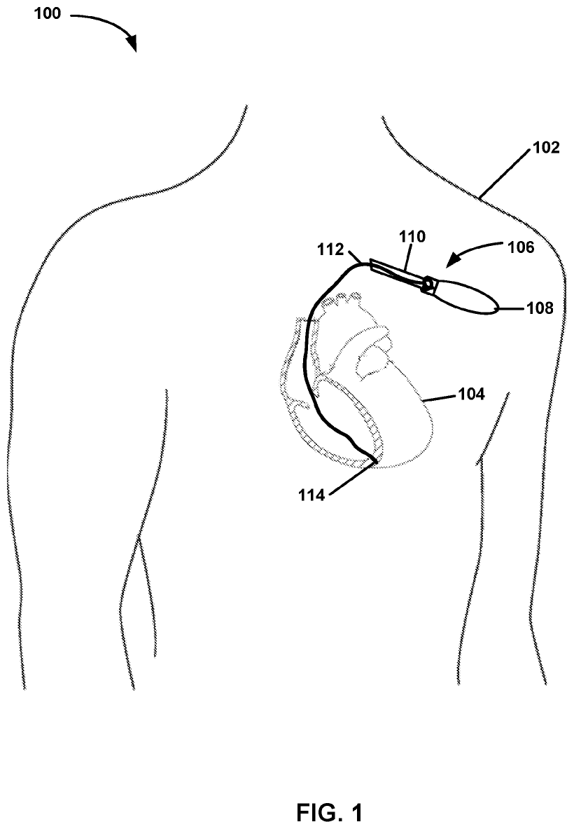

FIG. 1 is a conceptual diagram illustrating an implantable medical device system 100 including a lead management system 106. Medical device system 100 may include implantable medical device (IMD) 108, lead management system 106 including lead management device 110, and implantable medical lead 112 (also referred to herein as “electrical lead 112”). In some examples, as illustrated in FIG. 1, at least parts of medical device system 100 (e.g., IMD 108, lead management system 106, and electrical lead 112.) may be implanted within patient 102 (e.g., within a subcutaneous pocket in patient 102). A distal end 114 of lead 112 may be implanted at a target location (e.g., within heart 104 as illustrated in FIG. 1) within patient 102.

IMD 108 may include one or more devices configured to generate and deliver electrical energy (e.g., electrical stimulation, electrical therapy, or the like) to tissue of patient 102. The IMD 108 may be implanted within a subcutaneous pocket in patient 102. The subcutaneous pocket may be outside vasculature of patient 102. IMD 108 may deliver electrical energy to one or more target locations within patient 102 via one or more electrical leads 112. IMD 108 may be an implantable pulse generator (IPG), an implantable cardioverter defibrillator (ICD), a cardiac resynchronization therapy (CRT) device, or the like.

Electrical lead 112 may be connected to IMD 108 (e.g., to a distal end of IMD 108). In some examples, electrical lead 112 is fixedly attached to IMD 108, e.g., with no removable connection. In some examples, electrical lead 112 is removably attached to IMD 108, e.g., via a removable connector. Electrical lead 112 may transmit electrical signals between tissue of patient 102 and IMD 108. In some examples, as described in greater detail below, medical device system 100 may include a single electrical lead 112. In other examples, medical device system 100 may have two or more electrical leads 112. A clinician may insert electrical lead 112 into patient 102 at an insertion site (not pictured). The insertion site may be in the upper thorax or clavicular area of patient 102. In other examples, the insertion site may be at other locations on patient 102 (e.g., the groin, the neck, the abdomen, and the like). The medical professional may insert distal portion 114 of electrical lead 112 into vasculature of patient 102 at the insertion site and navigate electrical lead 112 to a target location (also referred to herein as “target treatment site”) within patient 102 through vasculature of patient 102 (e.g., within heart 114).

The clinician may navigate distal portion 114 of electrical lead 114 through the vasculature of patient 102 using one or more imaging techniques (e.g., X-ray imaging, fluoroscopy, ultrasound, or the like). Once the clinician determines that distal portion 114 of electrical lead 112 is at the target location, the clinician may implant the distal portion 114 at the target location via one or more fixation mechanisms attached to distal portion 114 of electrical lead 112. Depending on the anatomy of patient 102 and/or distance within the vasculature between the target location and the insertion site, a proximal portion of electrical lead 112 (also referred to as “excess portion) may be outside of the vasculature of patient 102.

Lead management system 106 includes one or more lead management devices 110 which may be disposed over IMD 108 and electrical lead 112 (e.g., the excess portion of electrical lead 112). Lead management device 110 may include one or more devices configured to control (e.g., inhibit or prevent) movement of electrical lead 112 and/or a length of electrical lead 112 within the vasculature of patient 102. In some examples lead management system 106 may include a single lead management device 110. In other examples, lead management system 106 may include two or more lead management devices 110. Lead management device 112 may be configured to at least partially retain electrical lead 112 and may include sheaths, holsters, expandable members, lead retention devices, or the like, as described in greater detail herein. Lead management device 110 may be permanently and/or removably connected to IMD 108 (e.g., to a distal end of IMD 108. Lead management device 110, IMD 108, and electrical lead 112 may be implanted within a subcutaneous pocket at the insertion site. In some examples, while implanted, lead management device 110 may isolate IMD 108 and/or the excess portions of electrical lead 112 from tissue of patient 102 around the subcutaneous pocket and prevent tissue ingrowth around IMD 108 and/or electrical lead 112.

When the clinician decides to remove IMD 108 and/or electrical lead 112 from patient 102, the clinician may re-open the subcutaneous pocket and remove lead management device 110 containing IMD 108 and electrical lead 112 without necessitating removal of ingrown tissue around IMD 108 and/or electrical lead 112. In some examples, electrical lead 112 may remain implanted within patient 102 and the clinician may replace IMD 108 of medical device system 100 with another implantable medical device. In some examples, where lead management system 106 includes two or more lead management devices 110, the clinician may remove and/or access one or more of lead management devices 110 without removing another lead management device 110, IMD 108, and/or electrical lead 112.

Medical system 100 may also include one or more computing devices and/or systems (not pictured) in communications with IMD 108. The one or more computing devices and/or systems may be operated by a user (e.g., by patient 102 and/or the clinician) and may transmit instructions to IMD 108. In some examples, the one or more computing devices and/or systems may receive patient information detected by IMD 108 (e.g., via electrical lead 112).

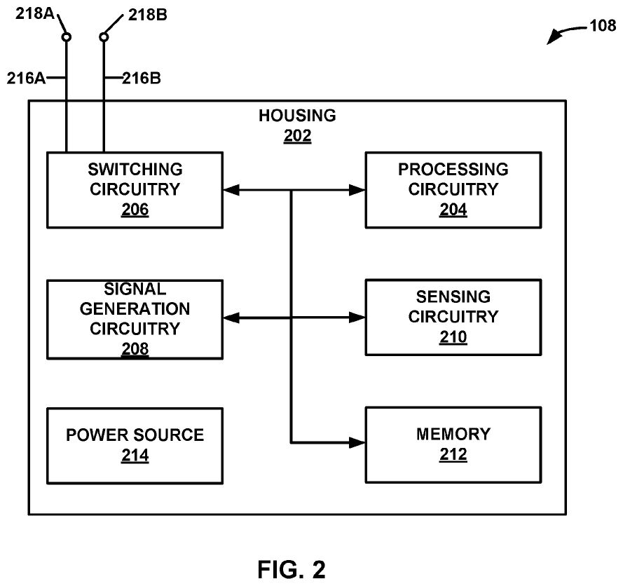

FIG. 2 is a block diagram illustrating an example configuration of an IMD 108 of the medical device system 100 of FIG. 1. IMD 108 may include processing circuitry 204, switching circuitry 206, signal generation circuitry 208, sensing circuitry 210, memory 212, power source 214, and electrodes 218A-B (also referred to as “electrodes 218”) connected to conductors 216 A-B (also referred to as “conductors 216”). Conductors 216 and electrodes 218 may be disposed within electrical lead 112 (not pictured). While FIG. 2 illustrates IMD 108 having two electrodes 218, other example IMDs may include three or more electrodes, each electrode connected to a corresponding conductor. In some examples, IMD 108 may have additional components (e.g., communications circuitry or the like). In some examples, as illustrated in FIG. 2, the components of IMD 108 may be contained within housing 202. In some examples electrodes 218 may be disposed in distal end 114 of electrical lead 112.

The various circuitry may be, or include, programmable or fixed function circuitry configured to perform the functions attributed to respective circuitry. Memory 212 may store computer-readable instructions that, when executed by processing circuitry 204, cause computing system 112 and/or implantable medical lead 104 to perform various functions. Memory 212 may be a storage device or other non-transitory medium.

Signal generation circuitry 208 is configured to generate electrical signals (e.g., stimulation signals such as cardiac pacing pulses). Signal generation circuitry 208 may include, as examples, current or voltage sources, capacitors, charge pumps, or other signal generation circuitry. Switching circuitry 206 is coupled to electrodes 218 and may include one or more switch arrays, one or more multiplexers, one or more switches (e.g., a switch matrix or other collection of switches), one or more transistors, or other electrical circuitry. Switching circuitry 206 is configured to direct electrical signals from signal generation circuitry 208 to a selected combination of electrodes 218, having selected polarities, e.g., to selectively deliver electrical signals to one or more target locations within patient 102. In some examples, Signal generation circuitry 208 and switching circuitry 206 may be configured to selectively deliver electrical signals to one or more chambers of heart 104 of patient 102 to deliver cardiac pacing pulses to heart 104.

Switching circuitry 206 may also selectively couple sensing circuitry 210 to selected combinations of electrodes 218 to selectively sense the electrical activity in one or more target locations within patient 102. Sensing circuitry 210 may include filters, amplifiers, analog-to-digital converts, or other circuitry configured to sense electrical signals (e.g., cardiac electrical signals) via electrodes 218. For example, switching circuitry 206 may couple each of electrodes 218 to respective sensing channels provided by sensing circuitry 210 to sense the electrical signals in the corresponding target locations. Sensing circuitry 210 may sense electrical signals via electrodes 218 and store the sensed electrical signals within memory 212.

Processing circuitry 204 may include any one or more of a microprocessor, a controller, a digital signal processor (DSP), an application specific integrated circuitry (ASIC), a field-programmable gate array (FPGA), discrete logic circuitry, or any other processing circuitry configured to provide the functions attribute to processing circuitry 204 herein may be embodied as firmware, hardware, software, or any combination thereof.

Processing circuitry 204 may retrieve and execute instructions from memory 212 and transmit instructions to switching circuitry 206, signal generation circuitry, and sensing circuitry 210 to perform the functions described above.

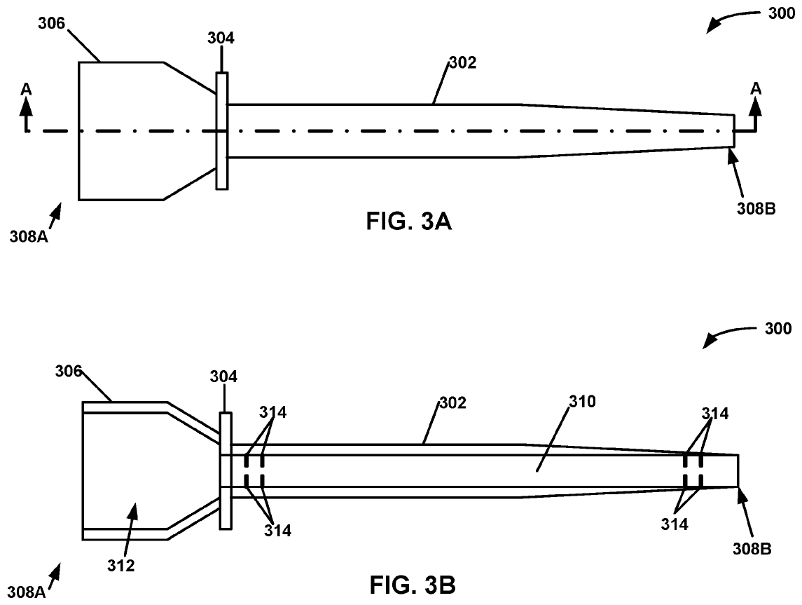

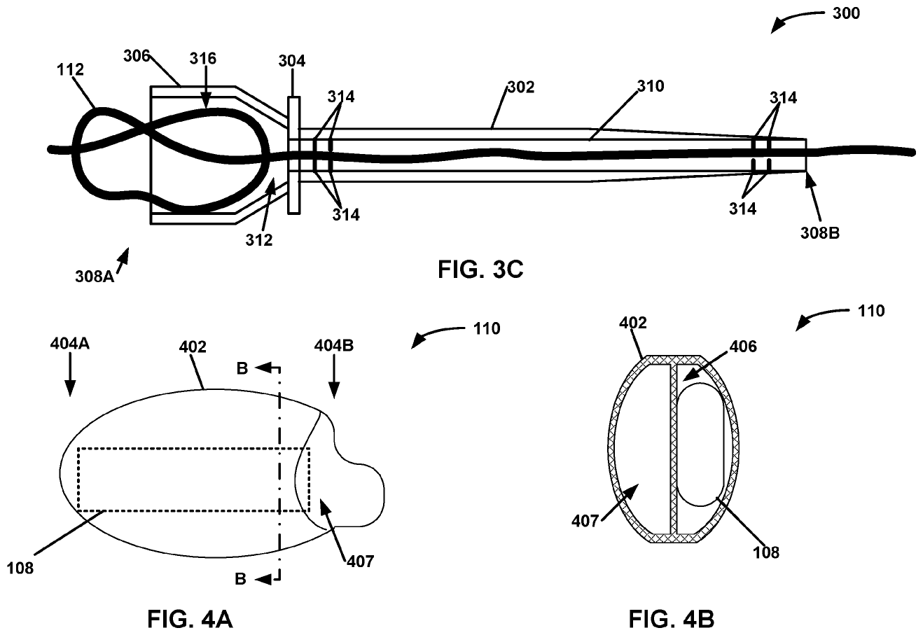

FIG. 3A is a conceptual diagram illustrating an example sheath 300 of an example lead management system 106. FIG. 3B is a conceptual diagram illustrating a cross-section diagram of sheath 300 of FIG. 3A taken along line A-A. FIG. 3C is a conceptual diagram illustrating sheath 300 of FIG. 3B with electrical lead 112. In some examples, sheath 300 is configured as lead management device 110 of lead management system 106 and may be connected to IMD 108 (e.g., to a distal end of IMD 108). In some examples, lead management system 106 includes one or more lead management devices 110 in addition to sheath 300. Sheath 300 may be connected to another lead management device 110 (e.g., a retainer, a holster, or the like). Sheath 300 may be permanently or removably connected to IMD 108 and/or lead management device 110.

Sheath 300 includes a shaft 302 extending from proximal end 308A to distal end 308B of sheath 300. Sheath 300 may include a flange 304 disposed on shaft 302 (e.g., on a proximal portion of shaft 302 and/or near proximal end 308A of sheath 300). In some examples, sheath 300 may include chamber 306 disposed on proximal end 308A and in communication with shaft 302. Sheath may include biocompatible materials including, but are not limited to silicone, polyurethanes (e.g., Carbothane), Pellethane, Elastane, PEBAX, expanded Polytetrafluoroethylene (EPTFE), or the like.

Shaft 302 defines an inner lumen that is configured to retain electrical lead 112. At least a portion of shaft 302 (e.g., a distal portion of shaft 302) may be disposed within the vasculature of patient 102. In some examples, as illustrated in FIG. 3A, at least a portion of shaft 302 may be tapered, e.g., to facilitate insertion of shaft 302 into the vasculature. A lubricious material may be applied over at least a part of an outer surface of shaft 302, e.g., to facilitate insertion of shaft 302 into the vasculature. The lubricious material may include, but are not limited to, silicone oil, Polytetrafluoroethylene (PTFE), a calcium carbonate coating, or Ethylene tetrafluoroethylene (ETFE). In some examples, an anti-infection material may be applied over shaft 302 and/or within shaft 302. The anti-infection material may include, but is not limited to, antimicrobial agents such as minocycline and/or rifampin. In some examples, an anti-bleeding and anti-coagulation material may be applied over shaft 302 and/or within shaft 302. The anti-bleeding and anti-coagulation material may include but are not limited to, Alginate, or Tranexamic Acid.

Flange 304 may have an outer diameter that is greater than a maximum outer diameter of shaft 302. Flange 304 may anchor sheath 300 within body of patient 102, e.g., within the vasculature of patient 102. In some examples, flange 304 interacts with a wall of a blood vessel to prevent further movement of sheath 300 and/or shaft 302 into the blood vessel. In some examples, flange 304 may aid the clinician in manipulation of sheath 300.

Chamber 306 may be permanently or removably connected to shaft 302. Chamber 306 defines an inner recess that may retain at least a portion of the excess portion of electrical lead 112 (e.g., in a storage configuration). In some examples, chamber 306 may include one or more fixation features (not pictured) configured to connect chamber 306 to IMD 108 (e.g., to a distal end of IMD 108) and/or lead management device 110. In some examples, chamber 306 may taper from the outer diameter of shaft 302 to a maximum outer diameter of chamber 306. Chamber 306 may be tapered to facilitate insertion of sheath 300 through the tissue of patient 102.

As illustrated in FIG. 3B, shaft 302 defines inner lumen 310 and chamber 306 defines inner recess 312 in communication with inner lumen 310. Sheath 300 may define openings at proximal end 308A and distal end 308B to allow for insertion and/or retraction of electrical lead 112 through sheath 300 (e.g., through inner lumen 310).

Inner lumen 310 may extend from inner recess 312 to distal end 308A of sheath 300. Inner lumen 310 may retain electrical lead 112 and may allow insertion and/or retraction of electrical lead 112 within inner lumen 310. In some examples, an inner surface of shaft 302 defining inner lumen 310 may include a lubricious material and/or an anti-infection material.

Shaft 302 may include one or more valves 314 disposed within inner lumen 310. Valves 314 may be disposed in the proximal portion and/or a distal portion of inner lumen 310. Valves 314 may reduce and/or prevent flow of fluids (e.g., blood) through inner lumen 310. In some examples, valve 314 may seal around electrical lead 112 disposed within inner lumen 310 to prevent fluid flow. Valves 314 may create hemostasis around an insertion point of sheath 300 into the blood vessel.

An excess portion 316 of electrical lead 112 may be disposed within inner recess 312 and electrical lead 112 may extend through inner lumen 310 and out of distal end 308B of sheath 300. Electrical lead 112 may be sized such that valves 314 seals around electrical lead 112 with minimal interference.

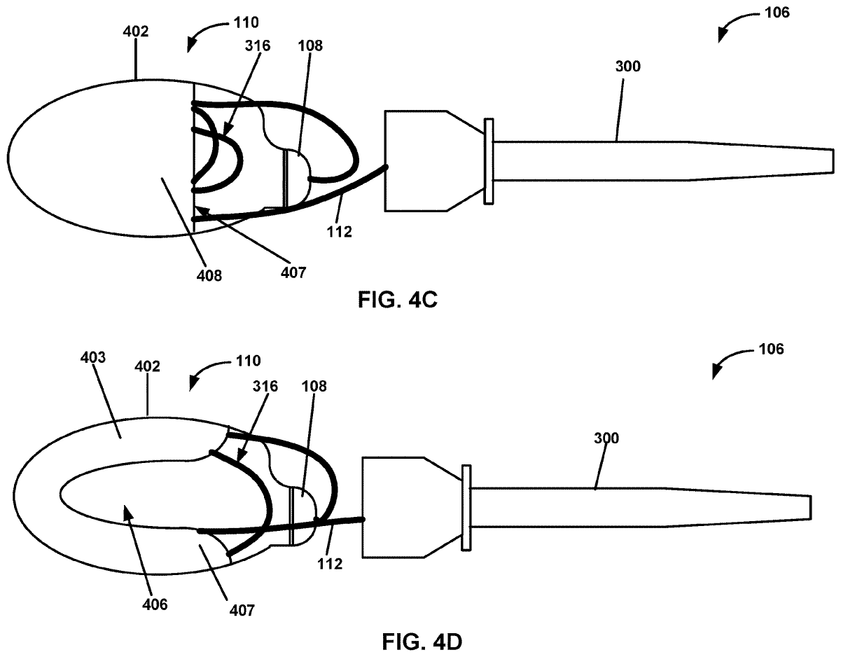

FIG. 4A is a conceptual diagram illustrating another example lead management device 110. FIG. 4B is a conceptual diagram illustrating a cross-section view of the lead management device 110 of FIG. 4A taken along line B-B. FIG. 4C is a conceptual diagram illustrating an example medical device system 106 with the lead management device 110 of FIG. 4A. FIG. 4D is a conceptual diagram illustrating another example medical device system 106 with another example lead management device 100 of FIG. 4A. In some examples, the lead management device 110 illustrated in FIGS. 4A-4D may be separate from IMD 108. In some examples, lead management device 110 may be integral to IMD 108 (e.g., is a part of housing 202 of IMD 108).

Lead management device 110 may be a holster 402 extending from a proximal end 404A to a distal end 404B. As illustrated in FIG. 4B, holster 402 may define a first pocket 406 and a second pocket 407. First pocket 406 and second pocket 407 may be arranged in an over-under configuration, a side-by-side configuration (e.g., as illustrated in FIG. 4B), or the like. IMD 108 may be disposed within first pocket 406. In some examples, IMD 108 is entirely disposed within first pocket 406. In other examples, IMD 108 is partially disposed within first pocket 406.

Excess portion 316 of electrical lead 112 may be disposed within second pocket 407. In some examples, excess portion 316 is arranged in a storage configuration and disposed within second pocket 407. While in the storage configuration, excess portion 316 may be stored in one or more coils without pinching, crimping, and/or twisting of the one or more coils.

In some examples, as illustrated in FIG. 4C, second pocket 407 may be defined by holster cover 408. Holster cover 408 may fully cover at least a portion of second pocket 407 and may not include any recesses and/or openings in holster cover 408. In some examples, holster cover 408 may be expandable and may retain excess portions 316 of electrical leads 112 of a range of lengths and lead diameters. Excess portions 316 of electrical lead 112 may be up to 30 centimeters (cm) in length. The lead diameter of electrical lead 112 may be between about 5 French (Fr) and about 9 Fr (e.g., between about 0.06 inches and about 0.12 inches, between about 1.5 millimeters (mm) and about 3 mm).

In other examples, as illustrated in FIG. 4C, second pocket 407 may be defined by holster body 402, which defines recess 406 into second pocket 407. In such examples, the clinician may use recess 406 to visually inspect excess portion 316 within second pocket 407 and/or may manipulate portions of excess portion 316 without removing excess portion 316 from second pocket 407. Recess 406 may also allow for easier removal of excess portion 316 from holster 402. Holster 402 and/or holster cover 408 may include biocompatible polymers including, but is not limited to, silicone, polyurethane, Pellethane, elastane, PEBAX, or the like.

As illustrated in FIGS. 4C and 4D, a distal portion of holster 402 may be connected to a proximal end of sheath 400. In some examples, sheath 400 may be connected to an expandable member that may be disposed over holster 402 containing IMD 108 and excess portion 316. In some examples, the expandable member may be independent from sheath 400 and may be disposed over holster 402.

Examples of the expandable member are described in the U.S. Provisional Patent Application entitled “Temporary Pacing Lead Management System”, having Attorney Docket Number A0008411US01/2222-242USP1 and filed on the same day as this disclosure, the entirety of which is incorporated by reference herein. Other examples of the expandable member are described in the U.S. Provisional Patent Application entitled “Temporary Pacing Lead Management System with Expandable Member”, having Attorney Docket Number A0008375US01/2222-254USP1 and filed on the same day as this disclosure, the entirety of which is incorporated by reference herein.

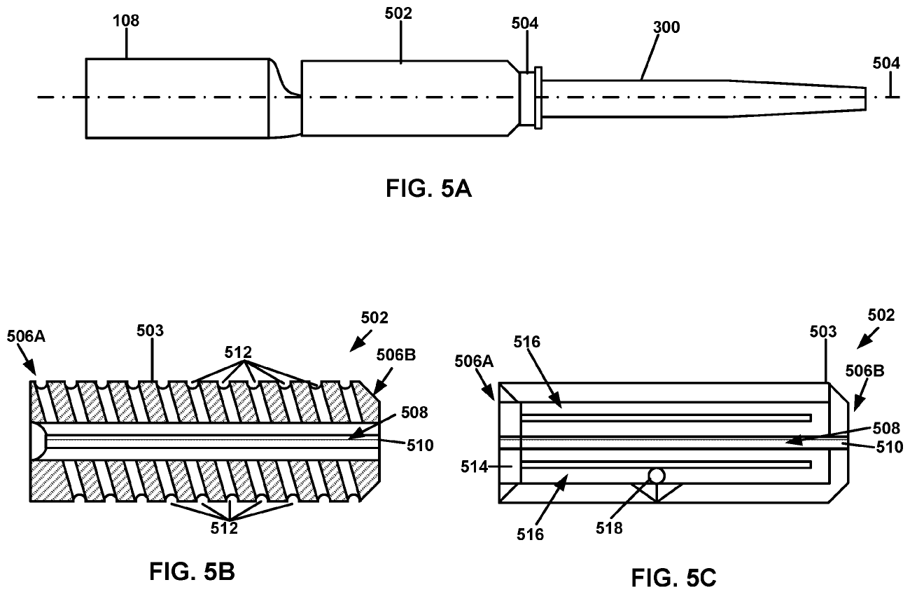

FIG. 5A is a conceptual diagram illustrating another example lead management system 106. Lead management system 106, as illustrated in FIG. 5A, may include IMD 108, retainer 502, and sheath 300 disposed along a longitudinal axis 504. Retainer 502 may be used alone or in any combination with other devices (e.g., sheath 300, holster 402, or the like) described herein.

Retainer 502 may include access feature 504 disposed on a distal end of retainer 502 and connected to a proximal end of sheath 300 (e.g., onto flange 304). Access feature 504 may allow for attachment and/or removal of sheath 300 from retainer 502. In some examples, access feature 504 may allow for access to an inner lumen of retainer 502 and/or inner lumen 312 of sheath 300 without detaching sheath 300 from retainer 502. Access feature 504 may include one or more features that hinge open and allows access to the inner lumen of retainer 502 and/or inner lumen 312.

FIG. 5B is a conceptual diagram illustrating an example retainer 502 of FIG. 5A. Retainer 502 may include a retainer body 503 and a central groove 508 extending from a proximal end 506B of retainer body 503 to a distal end 506B of retainer body 503. In some examples, retainer 502 may include materials including, but are not limited to, silicone, Polyurethane, Carbothane, Pellethane, Elastane, PEBAX, or the like.

Central groove 508 may retain electrical lead 112 within at least a portion of central groove 508. Retainer body 503 may define aperture 510 (or “opening 510”) at distal end 506B. Aperture 510 may be in communication with inner lumen 310 of sheath 300 and may allow for insertion of electrical lead 112 from central groove 508 and into inner lumen 310. In some examples, a dilator, a guidewire, or the like may be disposed within central groove 508 to facilitate navigation of retainer 502 and/or medical device system 106 within tissue of patient 102.

Retainer body 503 may define a plurality of grooves 512 extending around an outer surface of retainer body 503. In some examples, as illustrated in FIG. 5B, grooves 512 may form a helical pattern around the outer surface of retainer body 503. Electrical lead 112 may be disposed within grooves 512 (e.g., via snap-fitting, press-fitting, or the like). The clinician may dispose electrical lead 112 within one or more of grooves 512 depending on a length of excess portion 316.

In some examples, the clinician may first advance a distal portion of electrical lead 112 through aperture 510 and through vasculature of patient 102. The clinician may then secure excess portion 316 in grooves 512 until a user-determined amount of electrical lead 112 is secured within retainer 502. The clinician may then dispose an expandable member over retainer 502 and excess portion 316. The expandable member may isolate retainer 502 and excess portion 316 from the tissue of patient 102 and prevent tissue growth around retainer 502 and/or excess portion 316. In some examples, expandable member may include an anti-infection material, e.g., disposed over an outer surface and/or inner surface of the expandable member and/or within the expandable member. The anti-infection material may include, but are not limited to, minocycline or rifampin. In some examples, the expandable member may include an anti-bleeding and anti-coagulation material, e.g., applied over an outer surface of the expandable member. The anti-bleeding and anti-coagulation material may include but are not limited to, Alginate, or Tranexamic Acid.

FIG. 5C is a conceptual diagram illustrating another example retainer 502 of FIG. 5A. Retainer body 503 defines grooves 516 around central groove 508. In some examples, as illustrated in FIG. 5C, grooves 516 may be parallel to central groove 508. Grooves 516 may be arranged in a bobbin-like configuration.

Electrical lead 112 may be disposed within grooves 516 (e.g., via snap-fitting, press-fitting, or the like). The clinician may dispose electrical lead 112 within one or more of grooves 516, depending on a length of excess portion 316. In some examples, as illustrated in FIG. 5C, retainer body 503 may define aperture 518 within one of grooves 516. Electrical lead 112 may be inserted into grooves 516 via aperture 518.

Retainer body 503 may also define ramp 514 at proximal end 506A of retainer 502. Ramp 514 may interact with IMD 108 and/or one or more lead management devices 106 to connect retainer 502 to IMD 108 and/or lead management devices 106.

In some examples, the clinician may first advance a distal portion of electrical lead 112 through aperture 510 and/or aperture 518 and through vasculature of patient 102.

The clinician may then secure excess portion 316 in grooves 516 until a user-determined amount of electrical lead 112 is secured within retainer 502. The clinician may then dispose an expandable member over retainer 502 and excess portion 316.

The expandable member, as described in FIGS. 4A-D, or FIGS. 5A-C, may isolate lead management system 106 from tissue of patient 102. In some examples, expandable member prevents tissue growth around IMD 108, excess portion 316 and/or lead management device 110 of lead management system 106, thereby facilitating removal of medical device system 100 from patient 102.

The shape and/or dimensions of the expandable member may vary based on the shape and/or dimensions of lead management system 106. The expandable member may be transparent or translucent to allow the clinician to visually inspect disposition of IMD 108 and/or excess portion 316 within lead management device 110. The expandable member may be disposed over at least a portion of lead management device 110, sheath 300, IMD 108, and/or excess portion 316. In some examples, the outer surface of the expandable member may include a lubricious material. The lubricious material may improve navigability of lead management system 106 into and/or out of tissue of patient 102. The expandable material may include a biocompatible polymer including silicone.

In some examples, the expandable member may be disposed on a distal portion of lead management device 110 (e.g., near distal end 404B, near distal end 506B, or the like) in a pre-deployed configuration. The clinician may then transition the expandable member to a deployed configuration along a longitudinal axis of lead management system 106 to dispose the expandable member around lead management system 106 (e.g., around an entirety of IMD 106, excess portion 316 of electrical lead 112, lead management device(s) 110, or the like). In some examples, the clinician may roll the expandable member over lead management system 106 to transition the expandable member from the pre-deployed configuration to the deployed configuration.

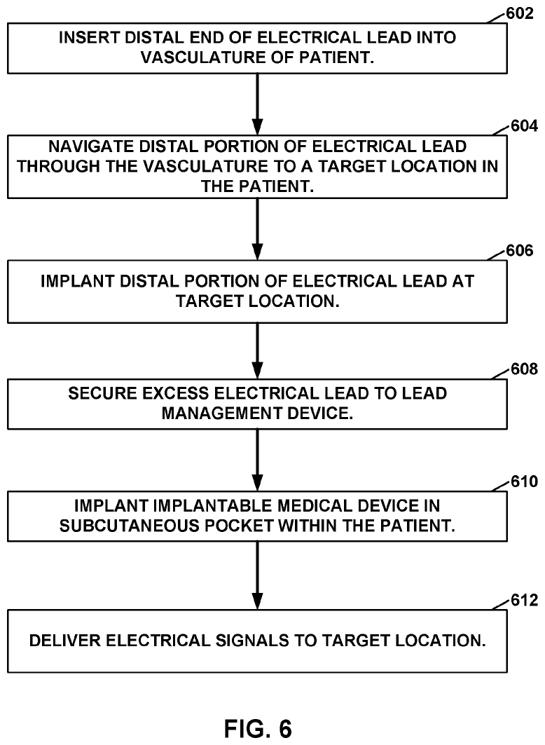

FIG. 6 is a flowchart illustrating an example process of implanting an medical device system in a patient. A clinician may insert distal end 114 of an electrical lead 112 into vasculature of patient 102 (602). The clinician may make an insertion at an insertion site on patient 102. The insertion may extend from the skin of patient 102 into vasculature of patient 102, e.g., via a blood vessel of patient 102. Once the clinician makes the incision, the clinician may insert distal end 114 of electrical lead 112 through the incision and into the vasculature. In some examples, the clinician may dispose electrical lead 112 within a sheath (e.g., sheath 300), insert sheath 300 and electrical lead 112 into the vasculature, and anchor sheath 300 in tissue of patient 102 (e.g., via flanges 304). In some examples, the clinician may use a dilator, a guidewire, or other device to insert distal end 114 and/or sheath 300 into the vasculature of patient 102.

The clinician may navigate electrical lead 112 through the vasculature to a target location (also referred to as “target treatment site”) within patient 102 (604). The target location may be any location within vasculature of patient 102 that the clinician wants to deliver electrical energy to. In some examples, the target location may include wall tissue (e.g., at the ventricular apex, the interventricular septum, or the Triangle of Koch) of one or more chambers of heart 104 of patient 102. The clinician may navigate electrical lead 112 to the target location via one or more imaging techniques (e.g., X-ray imaging, fluoroscopy, ultrasound, or the like). In some examples, the clinician may navigate electrical lead 112 via one or more radiopaque markers disposed on electrical lead 112.

The clinician may implant distal end 114 of electrical lead 114 at the target location (606). The clinician may implant distal end 114 of electrical lead 112 via one or more fixation features disposed on distal end 114. Fixation features may include, but are not limited, penetrator(s) (e.g., a helical penetrator), tines, adhesives, or the like.

The clinician may dispose IMD 108 and excess portion 306 of electrical lead 112 within lead management system 106 (608). In some examples, if lead management system 106 includes sheath 300, the clinician may arrange excess portion 316 into a storage configuration and retain excess portion 312 into inner recess 312 of sheath 300.

In some examples, if lead management system 106 includes holster 402, the clinician may dispose IMD 108 into first pocket 406 and excess portion 316 into second pocket 407. The clinician may then connect holster 402 and/or IMD 108 to sheath 300. In some examples, after disposing IMD 108 and excess portion 316 into holster 402, the clinician may dispose an expandable member over holster 402 to isolate IMD 108 and excess portion 316 and connect holster 402 and/or IMD 108 to sheath 300.

In some examples, if lead management system 106 includes retainer 502, the clinician may dispose electrical lead 112 into sheath 300 (e.g., via aperture 510). The clinician may secure electrical lead 112 into central groove 508 of retainer 502. The clinician may then secure excess portion 316 of electrical lead 112 into one or more grooves (e.g., grooves 512, grooves 516, or the like) of retainer 502.

The clinician may implant lead management system 106 within a subcutaneous pocket in patient 102 (610). In some examples, the clinician may implant lead management system 106 within subcutaneous pocket 610 after disposing IMD 108 and excess portion 306 within one or more lead management devices 106 (e.g., sheath 300, holster 402, retainer 502, or the like). In some examples, the clinician may create subcutaneous pocket 610 and then dispose lead management system 106 within the subcutaneous pocket. In other examples, the clinician may create the subcutaneous pocket as a part of disposing lead management system 106 within tissue of patient 102.

The clinician may deliver electrical signals to target location (612). In some examples, the clinician may instruct IMD 108 to deliver electrical signals to target location via electrical lead 112 after implantation of IM 108 and electrical lead 112 within tissue of patient 102. In some examples, after implantation of IMD 108, IMD 108 may automatically transmit electrical signals to the target location.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

### Example 1

a system comprising: an electrical lead; an implantable medical device, wherein the electrical lead is configured to be electrically connected to the implantable medical device, and the implantable medical device is configured to deliver an electrical therapy to tissue of a patient via the electrical lead; a sheath configured to be at least partially disposed within vasculature of the patient and configured to receive the electrical lead such that a distal portion of the electrical lead is placed within the vasculature through the sheath; and a lead management device connected to the implantable medical device and the sheath, wherein the lead management device is configured to secure an excess portion of the electrical lead, and wherein the lead management device is configured to control movement of the electrical lead within the vasculature of the patient.

### Example 2

the system of example 1, wherein the lead management device is configured to secure the excess portion of the electrical lead in a storage configuration.

### Example 3

the system of example 2, wherein the lead management device includes one or more recesses configured to receive and secure the excess portion of the electrical lead in the storage configuration.

### Example 4

the system of any of examples 2 and 3, wherein the lead management device is configured to secure the excess portion of the lead in the storage configuration without twisting the electrical lead.

### Example 5

the system of any of examples 2-4, wherein the lead management device defines a pocket configured to retain the excess portion of the electrical lead in the storage configuration.

### Example 6

the system of example 5, wherein the lead management device comprises a holster defining a first pocket configured to retain the implantable medical device and a second pocket configured to retain the excess portion of the electrical lead in the storage configuration.

### Example 7

the system of example 6, wherein the second pocket defines a groove configured to secure the excess portion of the electrical lead.

### Example 8

the system of any of examples 6 and 7, wherein the first pocket and the second pocket are arranged in a side-by-side configuration.

### Example 9

the system of any of examples 1-4, wherein the lead management device comprises an elongated body defining: a central groove extending from a proximal end of the elongated body to a distal end of the elongated body, the central groove being configured to retain the electrical lead and dispose the electrical lead within the sheath; and a plurality of grooves disposed around the central groove and configured to retain the excess portion of the electrical lead.

### Example 10

the system of example 9, wherein the central groove defines an aperture at the distal end of the elongated body, wherein the aperture is connected to a proximal end of the sheath.

### Example 11

the system of any of examples 9 and 10, wherein the plurality of grooves form a helical recess around an outer surface of the elongated body.

### Example 12

the system of any of examples 9-11, wherein the plurality of grooves are parallel to the central groove.

### Example 13

the system of any of examples 9-12, wherein the distal end of the elongated body is removably connected to a proximal end of the sheath.

### Example 14

the system of any of examples 9-13, wherein the proximal end of the elongated body is configured to be connected to a distal end of the implantable medical device.

### Example 15

the system of any of examples 1-14, further comprising an expandable member disposed over the implantable medical device and the lead management device.

### Example 16

the system of example 15, wherein the expandable member is configured to isolate the implantable medical device and the lead management device from tissue of the patient.

### Example 17

the system of any of examples 15 and 16, wherein the expandable member further comprises an anti-infection material.

### Example 18

the system of any of examples 15-17, wherein the expandable member further comprises an anti-bleeding and anti-coagulation material.

### Example 19

the system of any of examples 1-18, wherein the sheath comprising anti-bleeding and anti-coagulation material disposed over an outer surface of the sheath.

### Example 20

the system of any of examples 1-19, wherein the implantable medical device and the lead management device are configured to be implanted into the patient in a subcutaneous pocket outside of the vasculature.

### Example 21

the system of any of examples 1-20, wherein the sheath anchors the electrical lead within the vasculature of the patient.

### Example 22

the system of any of examples 1-21, wherein the sheath comprises: an elongated body defining a sheath lumen configured to receive the electrical lead; and one or more valves disposed within the sheath lumen, the one or more valves configured to restrict flow of bodily fluids within the sheath lumen.

### Example 23

the system of example 22, wherein the one or more valves are disposed at a proximal end and a distal end of the sheath lumen.

### Example 24

the system of any of examples 1-23, wherein the sheath comprises a flange disposed on a proximal portion of the sheath, the flange being configured to anchor the sheath to a blood vessel wall within the vasculature of the patient.

### Example 25

the system of any of examples 1-24, wherein the lead management device is removably connected to the implantable medical device.

### Example 26

the system of any of examples 1-25, wherein the lead management device is permanently connected to the implantable medical device.

### Example 27

a lead management device comprising: a first pocket configured to retain an implantable medical device; and a second pocket configured to retain an excess portion of an electrical lead, wherein the electrical lead is electrically connected to the implantable medical device, and wherein the electrical lead is configured to be disposed within a vasculature of a patient and to deliver a medical therapy to tissue of the patient.

### Example 28

the device of example 27, wherein the first pocket and the second pocket are arranged in a side-by-side configuration.

### Example 29

the device of any of examples 27 and 28, wherein the lead management device is configured to control movement of the electrical lead within the vasculature of the patient by securing the excess portion of the electrical lead in a storage configuration within the second pocket.

### Example 30

the device of any of examples 27-29, wherein the lead management device is configured to secure the excess portion of the lead without twisting the electrical lead.

### Example 31

the device of any of examples 27-30, wherein the second pocket defines a groove configured to secure the excess portion of the electrical lead.

### Example 32

the device of any of examples 27-31, wherein the first pocket comprises a housing of the implantable medical device.

### Example 33

the device of any of examples 27-32, wherein a distal end of the lead management device is removably connected to a proximal end of a sheath configured to retain the electrical lead.

### Example 34

the device of any of examples 27-33, further comprising an expandable member disposed over the first pocket and the second pocket.

### Example 35

the device of any of examples 34, wherein the expandable member is configured to isolate the implantable medical device and the electrical lead from the tissue of the patient.

### Example 36

the device of any of examples 34 and 35, wherein the expandable member further comprises an anti-infection material.

### Example 37

the device of any of examples 34-36, wherein the expandable member further comprises an anti-bleeding and anti-coagulation material.

### Example 38

the device of any of examples 27-37, wherein the implantable medical device is removably connected to the first pocket.

### Example 39

the device of any of examples 27-38, wherein the implantable medical device is permanently connected to the first pocket.

### Example 40

a lead management device comprising: an elongated body defining: a central groove extending from a proximal end of the elongated body to a distal end of the elongated body, wherein the central groove is configured to retain a first portion of an electrical lead connected to an implantable medical device; and a plurality of grooves disposed around the center central groove and configured to retain a second portion of the electrical lead, wherein the lead management device is configured to control movement of the electrical lead.

### Example 41

the device of example 40, wherein the distal end of the elongated body is connected to a sheath configured to retain the electrical lead, and wherein the proximal end of the elongated body is connected to the implantable medical device.

### Example 42

the device of example 41, and wherein the central groove defines an aperture at the distal end of the elongated body connected to an opening on a proximal end of the sheath.

### Example 43

the device of any of examples 40-42, wherein the plurality of grooves form a helical recess around an outer surface of the elongated body.

### Example 44

the device of any of examples 40-43, wherein the plurality of grooves are parallel to the central groove.

### Example 45

the device of any of examples 40 44, further comprising one or more apertures extending from an outer surface of the elongated body to one or more of the plurality of grooves, wherein each of the one or more apertures is configured to allow for removal of at least a portion of the electrical lead through the aperture.

### Example 46

the device of any of examples 40-45, wherein the lead management device is configured to control a length of the electrical lead within the vasculature of the patient by securing the second portion of the electrical lead in a storage configuration within the plurality of grooves.

### Example 47

the device of example 46, wherein the lead management device is configured to secure the second portion of the electrical lead without twisting the electrical lead.

### Example 48

a method comprising: inserting a sheath and a distal portion of an electrical lead disposed within the sheath into vasculature of a patient; navigating the distal portion of the electrical lead to a target location within the vasculature of the patient; implanting the distal portion of the electrical lead in tissue at the target location; disposing an excess portion of the electrical lead within a lead management device, wherein the lead management device controls movement of the electrical lead; and delivering medical therapy to the tissue at the target location through the electrical lead.

### Example 49

the method of example 48, wherein the lead management device is connected to the implantable medical device and the sheath.

### Example 50

the method of any of examples 48 and 49, wherein disposing the excess portion of the electrical lead within the lead management device comprises securing the excess length of the electrical lead in the lead management device in a storage configuration.

### Example 51

the method of example 50, wherein disposing the excess portion of the electrical lead within the lead management device comprises securing the excess length of the electrical lead in the lead management device without twisting the electrical lead.

### Example 52

the method of any of examples 48-51, wherein the lead management device defines a first pocket and a second pocket, and disposing the excess portion of the electrical lead within the lead management device comprises: disposing the implantable medical device into the first pocket; disposing the excess length of the electrical lead into the second pocket; and inserting the electrical lead into the sheath.

### Example 53

the method of example 52, wherein the first pocket and the second pocket of the lead management device are arranged in a side-by-side configuration.

### Example 54

the method of any of examples 48-51, wherein the lead management device comprises an elongated body defining: a central groove extending from a proximal end of the elongated body to a distal end of the elongated body; and a plurality of grooves disposed around the central groove.

### Example 55

the method of example 54, wherein disposing the excess portion of the electrical lead within the lead management device comprises: inserting the electrical lead into the sheath; securing the electrical lead with the desired length into the central groove of the lead management device; and securing the excess portion of the electrical within one or more of the plurality of grooves of the lead management device.

### Example 56

the method of any of examples 54 and 55, wherein the plurality of grooves form a helical recess around an outer surface of the elongated body.

### Example 57

the method of any of examples 54-56, wherein the plurality of grooves are parallel to the central groove.

### Example 58

the method of any of examples 54-57, wherein the distal end of the elongated body is removably connected to a proximal end of the sheath.

### Example 59

the method of any of examples 48-58, where in the medical device system further comprises an expandable member disposed over the implantable medical device and the lead management device.

### Example 60

the method of example 59, wherein the expandable member is configured to isolate the implantable medical device and the lead management device from the tissue of the patient.

### Example 61

the method of any of examples 48-60, further comprising implanting the implantable medical device and the lead management device within a subcutaneous pocket in the tissue of the patient.

### Example 62

the method of any of examples 48-61, wherein the sheath anchors the distal portion of the electrical lead within the vasculature of the patient.

### Example 63

the method of any of examples 48-62, wherein the sheath comprises: an elongated body defining a sheath lumen; and one or more valves disposed within the sheath lumen, the one or more valves configured to restrict flow of bodily fluids within the sheath lumen.

Various examples have been described herein. Any combination of the described operations or functions is contemplated. These and other examples are within the scope of the following claims.

1: A system comprising:
an electrical lead;
an implantable medical device, wherein the electrical lead is electrically connected to the implantable medical device, and the implantable medical device is configured to deliver an electrical therapy to tissue of a patient via the electrical lead;
a sheath configured to be at least partially disposed within vasculature of the patient and configured to receive the electrical lead such that a distal portion of the electrical lead is placed within the vasculature through the sheath; and
a lead management device connected to the implantable medical device and the sheath, wherein the lead management device is configured to secure an excess portion of the electrical lead, and wherein the lead management device is configured to control movement of the electrical lead within the vasculature of the patient.

2: The system of claim 1, wherein the lead management device includes one or more recesses configured to receive and secure the excess portion of the electrical lead in a storage configuration.

3: The system of claim 1, wherein the lead management device defines a pocket configured to retain the excess portion of the electrical lead in the storage configuration.

4: The system of claim 3, wherein the lead management device comprises a holster defining a first pocket configured to retain the implantable medical device and a second pocket configured to retain the excess portion of the electrical lead in the storage configuration.

5: The system of claim 4, wherein the second pocket defines a groove configured to secure the excess portion of the electrical lead.

6: The system of claim 1, wherein the lead management device comprises an elongated body defining:
a central groove extending from a proximal end of the elongated body to a distal end of the elongated body, the central groove being configured to retain the electrical lead and dispose the electrical lead within the sheath; and
a plurality of grooves disposed around the central groove and configured to retain the excess portion of the electrical lead.

7: The system of claim 6, wherein the plurality of grooves form a helical recess around an outer surface of the elongated body.

8: The system of claim 6, wherein the plurality of grooves are parallel to the central groove.

9: The system of claim 1, further comprising an expandable member disposed over the implantable medical device and the lead management device, wherein the expandable member is configured to isolate the implantable medical device and the lead management device from tissue of the patient.

10-12. (canceled)

13: The system of claim 1, wherein the sheath anchors the electrical lead within the vasculature of the patient.

14: The system of claim 1, wherein the sheath comprises:
an elongated body defining a sheath lumen configured to receive the electrical lead; and
one or more valves disposed within the sheath lumen, the one or more valves configured to restrict flow of bodily fluids within the sheath lumen.

15. (canceled)

16: A lead management device comprising:
a first pocket configured to retain an implantable medical device; and
a second pocket configured to retain an excess portion of an electrical lead, wherein the electrical lead is electrically connected to the implantable medical device, and wherein the electrical lead is configured to be disposed within a vasculature of a patient and to deliver a medical therapy to tissue of the patient.

17: The lead management device of claim 16, wherein the first pocket and the second pocket are arranged in a side-by-side configuration.

18: The lead management device of claim 16, wherein the lead management device is configured to control movement of the electrical lead within the vasculature of the patient by securing the excess portion of the electrical lead in a storage configuration within the second pocket.

19: The lead management device of claim 16, wherein the first pocket comprises a housing of the implantable medical device.

20: The lead management device of claim 16, further comprising an expandable member disposed over the first pocket and the second pocket, wherein the expandable member is configured to isolate the implantable medical device and the electrical lead from the tissue of the patient.

21: A lead management device comprising:
an elongated body defining:
a central groove extending from a proximal end of the elongated body to a distal end of the elongated body, wherein the central groove is configured to retain a first portion of an electrical lead connected to an implantable medical device; and
a plurality of grooves disposed around the central groove and configured to retain a second portion of the electrical lead,
wherein the lead management device is configured to control movement of the electrical lead.

22: The lead management device of claim 21, wherein the distal end of the elongated body is connected to a sheath configured to retain the electrical lead, and wherein the proximal end of the elongated body is connected to the implantable medical device.

23: The lead management device of claim 21, further comprising one or more apertures extending from an outer surface of the elongated body to one or more of the plurality of grooves, wherein each of the one or more apertures is configured to allow for removal of at least a portion of the electrical lead through the aperture.

24: The lead management device of claim 21, wherein the lead management device is configured to control a length of the electrical lead within the vasculature of the patient by securing the second portion of the electrical lead in a storage configuration within the plurality of grooves.
